(19) United States
(12) Patent Application Publication
(10) Pub. No.: US 2022/0143060 A1
(43) Pub. Date: May 12, 2022

(54) METHOD FOR TREATING INFECTIOUS DISEASES BY TARGETING NK CELL IMMUNE CHECKPOINT

(71) Applicant: INSTITUT PASTEUR OF SHANGHAI, CHINESE ACADEMY OF SCIENCES, Shanghai (CN)

(72) Inventors: Hong TANG, Shanghai (CN); Chao ZHANG, Shanghai (CN); Hairong CHEN, Shanghai (CN)

(73) Assignee: INSTITUT PASTEUR OF SHANGHAI, CHINESE ACADEMY OF SCIENCES, Shanghai (CN)

(21) Appl. No.: 17/441,661

(22) PCT Filed: Mar. 20, 2020
(86) PCT No.: PCT/CN2020/080365

§ 371 Date: Sep. 21, 2021

(30) Foreign Application Priority Data
Mar. 22, 2019 (CN) 201910219951.0

(51) Int. Cl. A61K 31/7072; A61K 31/7056; A61P 31/20

(52) U.S. Cl. CPC A61K 31/7072; A61P 31/20; A61K 31/7056

(57) ABSTRACT

The present disclosure relates to a method of preventing or treating an infectious disease in a subject, comprising the step of administering to the subject an antagonist or expression inhibitor for a natural killer (NK) cell immune checkpoint molecule. The present disclosure also relates to the use of an antagonist or expression inhibitor for an NK cell immune checkpoint molecular and pharmaceutical compositions comprising the same in the treatment of infectious diseases.

### PRIORITY

This application claims priority of CN application No.: 201910219951.0, filed Mar. 22, 2019, which is incorporated herein by reference in its entirety.

### TECHNICAL FIELD

The present disclosure relates to the field of immunotherapy. Specifically, the present disclosure relates to methods for preventing or treating infectious diseases by targeting NK cell immune checkpoints. The present disclosure also relates to the use of an antagonist or expression inhibitor for a NK cell immune checkpoint molecular and pharmaceutical compositions comprising the same in the treatment of infectious diseases.

### BACKGROUND ART

Infectious diseases caused by viral infections, including human immunodeficiency virus (HIV), hepatitis B virus (HBV), and hepatitis C virus (HCV), have spread globally. People of different genders, ages, and races have varying degrees of susceptibility to these viruses. For example, according to World Health Organization statistics, approximately 185 million people worldwide (about 3% of the total population) have been infected with HCV (Mohd Hanafiah, K., et al., Global epidemiology of hepatitis C virus infection: New estimates of age-specific antibody to HCV seroprevalence. Hepatology, 2013. 57(4): p. 1333-1342). It is estimated that there are currently more than 30 million HCV-infected patients in China, and the trend is increasing year by year.

During the acute phase of viral infection, if the virus cannot be eliminated in a quick manner, it often progresses to a chronic phase of infection. Among HCV infections, up to 80% of patients with acute infection cannot eliminate the virus and progress to chronic infection. Further major symptoms of chronic HCV infection include liver cirrhosis, portal hypertension, and liver cancer. According to data from WHO 2015, about 350,000 deaths are directly related to HCV each year, and about 27% of liver cirrhosis and about 25% of liver cancers are caused by HCV infection. In addition, HCV infection can cause a significant reduction in the quality of life of patients.

The standard regimen for HCV treatment is a combination of long-acting interferon injection and oral ribavirin. Taking into account the different HCV subtypes, about 50% of patients can achieve a sustained virological response (SVR) through treatment. In recent years, direct-acting antiviral drugs (DAA) have made great progress, and new DAA drugs are gradually being used in the clinic. However, the limitations of DAA drugs include: (1) drug resistance; (2) DAA drugs, like traditional ribavirin/long-acting interferon combination therapy, cannot effectively protect against reinfection and have limited efficacy in patients who have entered the middle or late stages of chronic infection; (3) at present, DAA drugs are of few types and expensive, which significantly increases the financial burden of patients.

Therefore, there remains a need in the art for efficient and economical treatment methods for infectious diseases caused by viral infections, including HCV, especially during chronic infection.

### SUMMARY OF THE INVENTION

The inventors have surprisingly found that by targeting immune checkpoint molecules expressed on NK cells (for example, using corresponding antagonists or expression inhibitors), it is possible to block, inhibit and/or reverse NK cell depletion in subjects with infectious diseases caused by viral infections. The above process further promotes the rapid elimination of the virus, thereby preventing or treating infectious diseases.

Accordingly, in one aspect, the present disclosure relates to a method for blocking, inhibiting, and/or reversing NK cell depletion in a subject, wherein the subject has or is at risk of suffering from an infectious disease caused by viral infection, the method comprising the step of administering to the subject an effective amount of an antagonist or expression inhibitor against an NK cell immune checkpoint molecule.

In another aspect, the present disclosure relates to a method of preventing or treating an infectious disease caused by viral infection in a subject, the method comprising the step of administering to the subject an effective amount of an antagonist or expression inhibitor against an NK cell immune checkpoint molecule.

The type of the above virus is not particularly limited, and may include any type of virus that can establish an infectious disease. In some embodiments, the virus may be selected from human immunodeficiency virus (HIV), hepatitis B virus (HBV), and hepatitis C virus (HCV). In some embodiments, the virus is HCV.

Human acquired immunodeficiency syndrome (AIDS) is a series of diseases caused by human immunodeficiency virus (HIV) infection. HIV includes HIV-1 and HIV-2, which is a retrovirus. HIV infects essential cells in the human immune system, such as helper T cells (especially CD4+ T cells), macrophages and dendritic cells. HIV infection causes a low level of CD4+ T cells through a variety of mechanisms, including apoptosis of infected T cells, apoptosis of uninfected adjacent cells, direct killing of virus infected cells, and killing of infected CD4+ T cells by CD8+ cytotoxic lymphocytes, etc. When the number of CD4+ T cells drops to below a critical level, cell-mediated immunity is lost, and the body is more susceptible to opportunistically infection, leading to the development of AIDS.

In most cases, HIV is a sexually transmitted infection that occurs through contact or transfer with blood, semen and vaginal fluids. In these bodily fluids, HIV exists as both free virus particles and viruses in infected immune cells. In addition, vertical transmission may occur between infected mothers and babies.

The initial stage after HIV infection is called acute HIV or primary HIV. Many people develop flu-like illness or mononucleosis-like illness 2-4 weeks after infection, while others have no obvious symptoms. The most common symptoms include fever, lymphadenopathy, inflammation of the throat, rash, headache, fatigue, and/or mouth and genital ulcers. Some patients also develop opportunistic infections at this stage. Symptoms vary in duration, but are usually for one or two weeks. Because of their non-specificity, these symptoms are often not recognized as a sign of HIV infection.

The second stage after the initial symptoms is called the clinical incubation phase/chronic infection phase, asymptomatic HIV or chronic HIV stage. The second stage of HIV infection can last for about 3-20 years (average about 8 years). Although usually with little or no symptoms at first, many people develop fever, weight loss, gastrointestinal problems, and muscle pain as this stage progresses, and 50%-70% of individuals also have persistent systemic lymphadenopathy. Most individuals infected with HIV-1 have a detectable viral load and eventually develop AIDS without treatment. Due to the progressive failure of the immune system, AIDS patients are at increasing risk of various viral infections and cancers. Without treatment, the average survival time for patients is 9 to 11 years.

Hepatitis B is a disease that affects the liver caused by infection with the hepatitis B virus (HBV). It can cause acute and chronic infections. About one third of the world's population is infected with HBV at some point in their lives, and about 343 million of them have a chronic infection. More than 750,000 people die each year from hepatitis B, of which about 300,000 are due to liver cancer. The disease can also affect other non-human apes. HBV transmission is mainly through exposure to infectious blood or blood-containing body fluids, which are 50 to 100 times more infectious than HIV. Possible forms of transmission include sexual contact, blood transfusion and infusion of other human blood products, re-use of contaminated needles and syringes, and vertical transmission from mother to child during childbirth. The virus can be detected within 30 to 60 days after infection and can persist and develop into chronic hepatitis B.

Acute HBV infection results in acute viral hepatitis, which begins with poor general health, loss of appetite, nausea, vomiting, general soreness, mild fever and dark urine, and then progresses to jaundice. The disease persists for several weeks and then is gradually improved in most affected people. A few people may have more severe liver disease, which is fulminant liver failure, and may die as a result. Acute infections may be completely asymptomatic and unrecognizable.

Chronic infections of HBV may be asymptomatic or associated with chronic inflammation of the liver (chronic hepatitis), leading to cirrhosis that lasts for years. This type of infection significantly increases the incidence of hepatocellular carcinoma. Across Europe, hepatitis B and C cause approximately 50% of hepatocellular carcinoma. These complications, including cirrhosis and liver cancer, cause 15% to 25% of patients with chronic HBV infection to die.

Hepatitis C is an infectious disease caused by the hepatitis C virus (HCV), which mainly affects the liver. According to World Health Organization statistics, about 185 million people (about 3% of the total population) are infected with HCV worldwide. According to estimates, there are currently more than 30 million HCV-infected patients in China, and the trend is increasing year by year. HCV is mainly transmitted through blood, and its transmission routes also include sexual transmission, mother-to-child transmission and so on. HCV is generally thought to infect humans and chimpanzees. Among HCV infections, up to 80% of patients with acute HCV infection cannot eliminate the virus and progress to chronic infection. Further major symptoms of chronic infections include liver cirrhosis, portal hypertension, and liver cancer. According to WHO data for 2015, approximately 350,000 deaths each year are directly related to HCV. In addition, HCV infection can cause a significant reduction in the quality of life of patients.

HCV infection causes acute symptoms in about 15% of cases. Symptoms are usually mild and include loss of appetite, fatigue, nausea, muscle or joint pain, and weight loss, as well as rare acute liver failure. Spontaneous clearance of the virus occurs in only 15%-20% of cases of HCV infection.

About 80% of individuals exposed to HCV are converted to chronic infection, which is defined as the presence of detectable viral replication for at least six months. Most people experience little or no symptoms during the first few years of a chronic infection. However, chronic infections can lead to liver cirrhosis or liver cancer years later. About 27% of liver cirrhosis and about 25% of liver cancer worldwide are caused by HCV infection. About 10-30% of people with HCV infection develop liver cirrhosis within 30 years. People with liver cirrhosis are 20 times more likely to develop hepatocellular carcinoma than normal people. The incidence of this conversion is 1-3% per year. In addition, liver cirrhosis may cause symptoms such as portal hypertension, ascites, easy bruising or bleeding, varicose veins, jaundice, and cognitive impairment syndrome (hepatic encephalopathy).

In some embodiments, the method described above is for preventing an infectious disease caused by a virus (e.g. HIV, HBV, or HCV) infection in a subject who is at risk of developing the infectious disease. For example, the subject has been in contact with a virus (e.g. HIV, HBV or HCV) infected person or carrier, such as through the transmission route of the corresponding virus, including blood transmission, sexual transmission, and mother-to-child transmission.

In some embodiments, the method described above is for treating an infectious disease in a subject caused by a viral (e.g. HIV, HBV or HCV) infection. In some embodiments, the infectious disease is in an acute infection phase. In other embodiments, the infectious disease is in a chronic infection phase.

For example, in some embodiments, the infectious disease is an HCV infection in a chronic infection phase.

As discussed above for various infectious diseases, viral infection is usually a process in which the pathogen and the host immune fight. Infection can be divided into acute and chronic infection stages. Acute infection is usually transient, the invasion of pathogen leading to the activation of immune system, the body quickly eliminating the pathogen and restoring homeostasis through native immunity or adaptive immune response; in chronic infection, the pathogen can persist through latent and escape host immunity. Chronic infection can affect the host by inducing cytopathy, inducing continuous inflammation, and establishing immune tolerance, leading to serious consequences such as decreased immunity, organ lesions and canceration, and even death. In this process, it is often accompanied by the depletion of immune effector cells such as T cells and NK cells.

In any embodiment of the above method, the NK cell immune checkpoint molecule may be selected from KR, NKG2A, TIGIT, and KLRG1.

KIR (Killer-cell immunoglobulin-like receptors) is a type I transmembrane glycoprotein family expressed on NK cells and a few T cells. They regulate the killing function of these cells by interacting with major histocompatibility (MHC) class I molecules (HLA-A in humans) expressed on nucleated cell types. Most KIRs are inhibitory, which means that their recognition of MHC molecules inhibits the cytotoxic activity of NK cells on which they are expressed. The initial expression of KIR on NK cells is random, but KIR expression changes during maturation of NK cells to achieve a balance between immune defense and self-tolerance. Because of its property of inhibiting NK cell activity, KIR is widely involved in viral infections, autoimmune diseases, and development of cancers.

NKG2A (CD94) is a member of the C-type lectin receptor family, which is a type II transmembrane protein. NKG2A is mainly expressed on the surface of NK cells and some CD8+ T cell subpopulations. NKG2A recognizes nonclassical MHC glycoprotein class I (HLA-E in humans and Qa-1 molecules in mice) and inhibits the cytotoxic activity of NK cells after binding to its ligand.

TIGIT (T cell immunoreceptor with Ig and ITIM domains) is an inhibitory immune receptor that present on some NK cells and T cells. TIGIT can bind to CD155 (PVR) on dendritic cells (DC), macrophages and other cells at a high affinity, and can also bind to CD112 (PVRL2) with at a lower affinity. TIGIT inhibits lymphocyte activity, while the DNAM-1 receptor is an activating receptor. TIGIT inhibits DNAM-1-mediated NK cell activation by competing with DNAM-1 for binding to the CD155 ligand. By blocking TIGIT, some functions of NK cells can be restored.

KLRG1 (Killer cell lectin-like receptor subfamily G member 1) is a lymphocyte co-suppression or immune checkpoint receptor, and is mainly expressed on late differentiated effector, effector and memory CD8+ T cells and NK cells. Its ligands are E-cadherin and N-cadherin with similar affinities, which are markers of epithelial cells and mesenchymal cells, respectively.

An “immune checkpoint molecule”, as understood by those skilled in the art, typically exists and functions as a ligand-receptor pair. Herein, immune checkpoint protein receptors and ligands thereof are collectively referred to as immune checkpoint molecules. For example, for NK cell immune checkpoint molecules, the receptor in a ligand-receptor pair is usually expressed in NK cells, while the corresponding ligand is expressed in other types of cells. NK cell activation and immune effector functions are inhibited by receptor-ligand interactions. Therefore, when referring to “immune checkpoint molecules”, both ligands and receptors are encompassed. For example, when referring to NKG2A, NKG2A and its ligands HLA-E (human) and Qa-1 (mouse) are encompassed.

In some embodiments, the antagonist is an antibody or antigen-binding fragment against the immune checkpoint molecule, including the receptor and its corresponding ligand.

As used herein, the term “antibody” refers to an immunoglobulin molecule comprising at least one antigen recognition site and capable of specifically binding to an antigen. The term “antibody” as referred to herein is understood in its broadest meaning and comprises monoclonal antibodies, polyclonal antibodies, antibody fragments, multispecific antibodies (e.g. bispecific antibodies) comprising at least two different antigen-binding domains. Antibodies also include murine-derived antibodies, chimeric antibodies, humanized antibodies, human antibodies, and antibodies of other origin. The antibodies of the present disclosure may be derived from any animal, including but not limited to immunoglobulin molecules of human, non-human primate, mouse or rat, etc. Antibodies may comprise further modifications, such as unnatural amino acid residues, mutations of Fc effector function, and mutations in glycosylation sites. Antibodies also include post-translationally modified antibodies, fusion proteins comprising the antigenic determinants of antibodies, and immunoglobulin molecules comprising any other modification of antigen recognition site, as long as these antibodies exhibit the desired biological activity.

The term “antigen-binding fragment” includes, but is not limited to: an Fab fragment having VL, CL, VH, and CH1 domains; an Fab′ fragment, which is a Fab fragment having one or more cysteine residues at the C-terminus of the CH1 domain; an Fd fragment having VI and CH1 domains; an Fd′ fragment having VH and CH1 domains and one or more cysteine residues at the C-terminus of the CH1 domain; an Fv fragment having VL and VH domains of a single arm of an antibody; a dAb fragment consisting of VH domains or VL domains; an isolated CDR region; a F(ab′)₂ fragment, which is a bivalent fragment of two Fab′ fragments connected by a disulfide bridge at the hinge region; a single-chain antibody molecule (e.g. single-chain Fv; scFv); “diabodies” with two antigen-binding sites, which comprises the heavy chain variable domain (VH) linked to the light chain variable domain (VL) in the same polypeptide chain; a “linear antibody” comprising a pair of tandem Fd segments (VH-CH1-VH-CH1) that together with a complementary light chain polypeptide form a pair of antigen-binding regions; and modified forms of any of the foregoing substances that retain antigen-binding activity.

In some embodiments of the method described above, the NK cell immune checkpoint molecule is NKG2A, and the antagonist is an antibody or antigen-binding fragment thereof directed against NKG2A or its ligand HLA-E. In some embodiments, the NK cell immune checkpoint molecule is KIR, and the antagonist is an antibody or antigen-binding fragment thereof directed against KIR or its ligand HLA-A. In some embodiments, the NK cell immune checkpoint molecule is TIGIT, and the antagonist is an antibody or antigen-binding fragment thereof directed against TIGIT or its ligand CD155 (PVR)/CD112 (PVRL2). In some embodiments, the NK cell immune checkpoint molecule is KLRG1, and the antagonist is an antibody or antigen-binding fragment thereof directed against KLRG1 or its ligand E-cadherin/N-cadherin.

In other embodiments, the antagonist is a soluble form of the corresponding ligand/receptor or fragment thereof of the immune checkpoint molecule. For example, where the NK cell immune checkpoint molecule is NKG2A, the antagonist may be a soluble form of NKG2A/HLA-E or a fragment thereof. Where the NK cell immune checkpoint molecule is KIR, the antagonist may be a soluble form of KIR/HLA-A or a fragment thereof. Where the NK cell immune checkpoint molecule is TIGIT, the antagonist can be a soluble form directed against TIGIT/CD155 (PVR)/CD112 (PVRL2) or a fragment thereof. In some embodiments, the NK cell immune checkpoint molecule is KLRG1, and the antagonist is a soluble form of KLRG1/E-cadherin/N-cadherin. The antagonist may also be a soluble form of a fusion protein comprising the receptor/ligand or a fragment thereof.

In some embodiments of the above method, the expression inhibitor is a microRNA or siRNA that inhibits the expression of a corresponding immune checkpoint molecule (including a receptor and its corresponding ligand), including various modified forms of microRNA or siRNA.

In some embodiments, the antagonist or expression inhibitor for a NK cell immune checkpoint molecule of the present disclosure is capable of promoting elimination of the virus.

In any embodiment of the method of the present disclosure, the subject includes, but is not limited to, non-human primates and humans. In some embodiments, the subject is a human.

In some embodiments, the method further comprises the step of administering one or more additional therapeutic agents to the subject.

In some embodiments, for example, the additional therapeutic agent is selected from an NK cell activation agent, such as an agonist for an NK cell activating receptor, an antagonist for an NK cell inhibitory receptor, or a cytokine or chemokine that activates NK cells. In some embodiments, the additional therapeutic agent is selected from a T cell (e.g. CD8+ T cell) activation agent, such as an agonist for a T cell activating receptor, an antagonist for a T cell inhibitory receptor, or a cytokine or chemokine that activates T cells. In other embodiments, the therapeutic agent is selected from direct-acting antiviral (DAA) drugs.

A number of DAA drugs have been developed in the field. For HBV infection, for example, currently commonly used DAA drugs include interferons, nucleoside analogs such as Lamivudine, Adefovir Dipivoxil, Telbivudine, Entecavir, Tenofovir Disoproxil, Clevudine and the like. For HCV infection, current DAA drugs include NS3/4A serine protease inhibitors, such as Telaprevir, Boceprevir, Simeprevir, and Asunaprevir; NS5B polymerase inhibitors, such as Sofosbuvir, Mericitabine (RG-7128), ACH-3422, and MK-3682; and NS5A replication complex protein inhibitors, including Daclatasvir.

For HIV infection, the currently commonly used therapy is a highly effective combination antiretroviral therapy using DAA drugs, also known as cocktail therapy. For example, a combination of two nucleoside reverse transcriptase inhibitors (NRTIs) and one non-nucleoside reverse transcriptase inhibitor (NNRTIs), or a combination of two NRTIs and one enhanced PIs (containing Ritonavir) is used.

Accordingly, in some embodiments, the virus is HBV, and the method comprises the step of administering to the subject a combination of the antagonist or expression inhibitor for an NK cell immune checkpoint and a DAA drug, with the DAA drug selected from interferon and nucleoside analogs such as Lamivudine, Adefovir Dipivoxil, Telbivudine, Entecavir, Tenofovir Disoproxil, Clevudine and the like.

In some embodiments, the virus is HCV, and the method comprises the step of administering to the subject a combination of the antagonist or expression inhibitor for an NK cell immune checkpoint and a DAA drug, with the DAA drug selected from Telaprevir, Boceprevir, Simeprevir, Asunaprevir, Sofosbuvir, Mericitabine (RG-7128), ACH-3422, MK-3682 and Daclatasvir.

In some embodiments, the antagonists or expression inhibitors of the present disclosure are for use in blocking, inhibiting and/or reversing NK cell depletion, or preventing or treating an infectious disease caused by viral infection in a subject who is not responsive or is tolerant to DAA drug treatment, with the DAA drugs, for example, as discussed above.

In one aspect, the disclosure relates to the use of an antagonist or expression inhibitor against an NK cell immune checkpoint molecule for blocking, inhibiting and/or reversing NK cell depletion in a subject, wherein the subject has or is at risk of an infectious disease caused by viral infection.

In another aspect, the present disclosure relates to the use of an antagonist or expression inhibitor against an NK cell immune checkpoint molecule in the manufacture of a pharmaceutical composition for blocking, inhibiting and/or reversing NK cell depletion in a subject, wherein the subject has or is at risk of an infectious disease caused by viral infection.

In one aspect, the present disclosure relates to the use of an antagonist or expression inhibitor against an NK cell immune checkpoint molecule for preventing or treating an infectious disease caused by viral infection in a subject.

In another aspect, the present disclosure relates to the use of an antagonist or expression inhibitor against an NK cell immune checkpoint molecule in the manufacture of a pharmaceutical composition for preventing or treating an infectious disease caused by viral infection in a subject.

In one aspect, the present disclosure relates to a pharmaceutical composition comprising an antagonist or expression inhibitor against an NK cell immune checkpoint molecule, and optionally one or more pharmaceutically acceptable carriers, excipients, and/or diluents, wherein the pharmaceutical composition is used for blocking, inhibiting and/or reversing NK cell depletion in a subject, wherein the subject has or is at risk of an infectious disease caused by viral infection.

In another aspect, the present disclosure relates to a pharmaceutical composition comprising an antagonist against an NK cell immune checkpoint molecule, and optionally one or more pharmaceutically acceptable carriers, excipients, and/or dilutions, wherein the pharmaceutical composition is used for preventing or treating an infectious disease caused by viral infection in a subject.

The phrase “pharmaceutically acceptable” means those compounds, materials, compositions and/or dosage forms suitable for use in contact with human and animal tissues within the scope of reasonable medical judgment without excessive toxicity, irritation, allergic response or other problems or complications and with a reasonable benefit/risk ratio. As used herein, the phrase “pharmaceutically acceptable carrier, excipient, and/or diluent” refers to a pharmaceutically acceptable material, composition, or vehicle, such as a liquid or solid filler, diluent, excipient, solvent, medium, encapsulating material, manufacturing aid, or solvent encapsulating material that maintains the stability, solubility, or activity of the antagonists of the present disclosure.

The pharmaceutical composition of the present disclosure can be administered through various routes, and is formulated according to different routes of administration. Preferably, the pharmaceutical composition is administered by a parenteral route, including but not limited to subcutaneous injection, intravenous injection (including bolus injection), intramuscular injection and intraarterial injection. Since administration of parenteral dosage forms usually bypasses the patient's natural defenses against contaminants, parenteral dosage forms are preferably sterile or capable of being sterilized before administration to the patient. Examples of parenteral dosage forms include, but are not limited to, solutions ready for injection, dry products ready to be dissolved or suspended in a pharmaceutically acceptable injection vehicle, suspensions ready for injection, controlled release parenteral dosage forms, and emulsions.

In some embodiments of the use and pharmaceutical composition described above, the virus may be selected from HIV, HBV, and HCV. For example, in some embodiments, the virus is HCV.

In some embodiments, the antagonist or expression inhibitor, or the pharmaceutical composition is for use in preventing an infectious disease caused by viral (e.g. HIV, HBV or HCV) infection in a subject who is at risk for the infectious disease, such as having been in contact with a virus (e.g. HIV, HBV or HCV)-infected or carrier.

In some embodiments, the antagonist or expression inhibitor, or the pharmaceutical composition is for use in treating an infectious disease caused by viral (e.g. HIV, HBV or HCV) infection in a subject. In some embodiments, the infectious disease is in an acute infection phase. In other embodiments, the infectious disease is in a chronic infection phase. For example, in some embodiments, the infectious disease is an HCV infection in a chronic infection phase.

In some embodiments of the above use, the NK cell immune checkpoint molecule may be selected from KIR, NKG2A, TIGIT, and KLRG1.

In some embodiments, the antagonist is an antibody or antigen-binding fragment against the immune checkpoint molecule, including its corresponding ligand. In some embodiments, the NK cell immune checkpoint molecule is NKG2A, and the antagonist is an antibody or antigen-binding fragment thereof directed against NKG2A or its ligand HLA-E. In some embodiments, the NK cell immune checkpoint molecule is KIR, and the antagonist is an antibody or antigen-binding fragment thereof directed against KIR or its ligand HLA-A. In some embodiments, the NK cell immune checkpoint molecule is TIGIT, and the antagonist is an antibody or antigen-binding fragment thereof directed against TIGIT or its ligand CD155 (PVR)/CD112 (PVRL2). In some embodiments, the NK cell immune checkpoint molecule is KLRG1, and the antagonist is an antibody or antigen-binding fragment thereof directed against KLRG1 or its ligand E-cadherin/N-cadherin.

In other embodiments, the antagonist is a soluble form of the corresponding ligand/receptor or fragment thereof of the immune checkpoint molecule. For example, where the NK cell immune checkpoint molecule is NKG2A, the antagonist may be a soluble form of NKG2A/HLA-E or a fragment thereof. Where the NK cell immune checkpoint molecule is KIR, the antagonist may be a soluble form of KIR/HLA-A or a fragment thereof. Where the NK cell immune checkpoint molecule is TIGIT, the antagonist can be a soluble form directed against TIGIT/CD155 (PVR)/CD112 (PVRL2) or a fragment thereof. In some embodiments, the NK cell immune checkpoint molecule is KLRG1, and the antagonist is a soluble form of KLRG1/E-cadherin/N-cadherin. The antagonist may also be a soluble form of a fusion protein comprising the receptor/ligand or a fragment thereof.

In some embodiments of the use described above, the expression inhibitor is a microRNA or siRNA that inhibits the expression of a corresponding immune checkpoint molecule (including a receptor and its corresponding ligand), including various modified forms of microRNA or siRNA.

In some embodiments, the antagonist or expression inhibitor for a NK cell immune checkpoint molecule or the pharmaceutical composition is for promoting elimination of the virus.

In any embodiment of the use described above, the subject includes, but is not limited to, non-human primates and humans. In some embodiments, the subject is a human.

In some embodiments, the antagonist or expression inhibitor is for use in combination with one or more additional therapeutic agents, or the pharmaceutical composition further comprises one or more additional therapeutic agents.

In some embodiments, the additional therapeutic agent is selected from an NK cell activation agent, such as an agonist for an NK cell activating receptor, an antagonist for an NK cell inhibitory receptor, or a cytokine or chemokine that activates NK cells. In some embodiments, the additional therapeutic agent is selected from a T cell (e.g. CD8+ T cell) activator, such as a T cell activating receptor agonist, a T cell inhibitory receptor antagonist, or a cytokine or chemokine that activates T cells. In other embodiments, the therapeutic agent is selected from a DAA drug. The DAA drug may be selected from, for example, those described above with respect to the method of the present disclosure.

In some embodiments, the antagonist or expression inhibitor, or the pharmaceutical composition of the disclosure is for use in blocking, inhibiting, and/or reversing NK cell depletion, or preventing or treating an infectious disease caused by viral infections in a subject who is not responsive or is tolerant to DAA drug treatment. The DAA drugs are as described above.

### DESCRIPTION OF THE DRAWINGS

FIG. 1 shows the establishment and confirmation of HCV infection models. FIG. 1A shows the results of the copy number of HCV genome in liver tissues in C/O-Tg mice and wild-type littermate control mice at different time points after tail vein infusion with HCV (n>6 at each time point) by qPCR, with the lower limit of detection being 100 copies/mg; FIG. 1B shows the results of the levels of IL-2, IL12p40 and IFN-γ in the serum of mice at different time points after infection by Luminex.

FIG. 2 shows the expression profile of T cell immune checkpoint molecules and the results of targeting these molecules during HCV infection. FIGS. 2A and 2B show the results of the expression levels of PD-1 and Tim-3 on the surface of CD8+ T cells in liver and peripheral blood at different time points after infection; FIG. 2C shows the results of the copy number of HCV in peripheral blood and liver tissue by qPCR after treatment of mice with PD-1 antibody or control antibody; FIG. 2D shows the results of the copy number of HCV in peripheral blood and liver tissue by qPCR after treatment of mice with PD-1 antibody in combination with Tim-3 antibody or control antibody.

FIG. 3 shows the results of immune tolerance and depletion of NK cells during HCV infection. FIG. 3A shows the results of the expression profiles of IFN-γ and CD107a of NK cells by flow cytometry after co-culturing liver NK cells with target cells Yac-1 at different time points after infection; FIG. 3B shows the results of expression of NK cell activating receptors Ly49D, Ly49H, and NKG2D; FIG. 3C shows the results of expression profiles of NK cell immune checkpoint molecules NKG2A, KLRG1, and TIGIT.

FIG. 4 shows the results of the expression levels of NKG2A in mice with different infection outcomes. FIG. 4A shows that C/O-Tg mice can be divided into self-limiting infection and chronic infection according to changes in serum virus copy number at one month after HCV infection; FIG. 4B shows the relationship between the serum virus copy number of mice and the expression profile of NKG2A on NK cells at one month after infection.

FIG. 5 shows the results of NKG2A antibodies inhibiting HCV to establish a chronic infection. FIG. 5A shows a schematic flow chart of the treatment of mice with antibodies, in which antibody administration was started one day before HCV infusion; FIG. 5B shows the results of copy numbers of virus in serum and liver tissues after one and two weeks of treatment with NKG2A antibody or control antibody; FIG. 5C shows the results of expression profiles of CD107a, granzyme B and IFN-γ of NK cells by flow cytometry after co-culture of liver NK cells with target cells Yac-1 after one and two weeks of treatment with NKG2A antibody or control antibody.

FIG. 6 shows the results of NKG2A antibodies promoting HCV elimination in established chronic infections. FIG. 6A shows a schematic flow chart of treating mice with antibodies, in which antibody administration was started two weeks after HCV infusion; FIG. 6B shows the results of copy number of virus in serum and liver tissues after four weeks of treatment with NKG2A antibody or control antibody; FIG. 6C shows the results of expression profiles of CD107a and granzyme B of NK cells by flow cytometry after co-culture of liver NK cells with target cells Yac-1 after four weeks of treatment with NKG2A antibody or control antibody; FIG. 6D shows the results of the function of HCV-specific CD8+ T cells by ELISPOT after four weeks of treatment with NKG2A antibody or control antibody.

FIG. 7 shows the results of Qa-1 antibodies inhibiting HCV to establish a chronic infection. FIG. 7A shows the results of Qa-1 mRNA level in liver tissue by qPCR at different time points of HCV infection; FIG. 7B shows the results of the copy numbers of virus in serum and liver tissues after two weeks of treatment with Qa-1 antibody or control antibody; FIG. 7C shows the results of the expression profiles of CD107a and IFN-γ of NK cells by flow cytometry after co-culture of liver NK cells with target cells Yac-1 after two weeks of treatment with Qa-1 antibody or control antibody; FIG. 7D shows the results of the function of HCV-specific CD8+ T cells by ELISPOT after two weeks of treatment with Qa-1 or control antibody.

FIG. 8 shows the results of siRNA interference with Qa-1 expression inhibiting HCV to establish a chronic infection. FIG. 8A shows a schematic flow chart of treating mice with Qa-1 siRNA or control siRNA, in which siRNA administration was started one day before HCV infusion; FIG. 8B shows the results of expression levels of Qa-1 mRNA of different cell components in liver tissue after two weeks of treatment with Qa-1 siRNA or control siRNA; FIG. 8C shows the results of the copy numbers of virus in serum and liver tissues after two weeks of treatment with Qa-1 siRNA or control siRNA; FIG. 8D shows the results of the expression profiles of CD107a and IFN-γ of NK cells by flow cytometry after co-culture of liver NK cells with target cells Yac-1 after two weeks of treatment with Qa-1 siRNA or control siRNA.

FIG. 9 shows the results of NKG2A antibodies function through NK cells. FIG. 9A shows the results of NK cell deletion; FIG. 9B shows the results of HCV-specific CD8+ T cell function by ELISPOT after two weeks of treatment with NKG2A antibody in the presence or deletion of NK cells; FIG. 9C shows the results of the copy numbers of virus in serum and liver tissues after two weeks of treatment with NKG2A antibody in the case of NK cell deletion or CD8+ T cell deletion.

### EXAMPLES

The present invention is further described below with reference to specific examples. The advantages and features of the present invention will become more apparent with the description. However, these examples are only exemplary and do not limit the scope of the present invention in any way. Those skilled in the art should understand that the details and forms of the technical solutions of the present invention can be modified or replaced without departing from the spirit and scope of the present invention, but these modifications and replacements fall within the protection scope of the present invention.

### Example 1. Establishment and Confirmation of HCV Mouse Model

During HCV infection, acute HCV infection is characterized by a significant delay in the onset of T cell response. In previous studies, human CD81 and OCLN liver-specific double transgenic mice (C/O-Tg mice) have been constructed on the background of ICR mice, which is able to support chronic HCV infection and mimic disease progressions such as immune tolerance, steatosis, liver fibrosis, and liver cirrhosis in chronic hepatitis C (Chen J, Zhao Y, Zhang C, Chen H, Feng J, et al. 2014. Persistent hepatitis C virus infections and hepatopathological manifestations in immune-competent humanized mice. Cell research 24:1050).

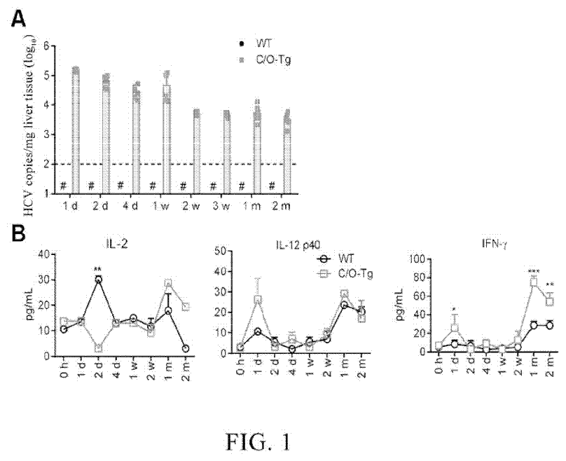

This mouse model of HCV infection was first reproduced and confirmed. HCV (J399EM, 1 mL, TCID₅₀=2×10⁷) was used for tail vein infusion in C/O-Tg mice and wild-type littermate control mice. Detection of the copy number of HCV genome in mouse liver at different time points after HCV infusion revealed HCV infection in C/O-Tg mice and the process of conversion from the acute infection phase to the chronic infection phase, while no HCV genome was detected in wild-type control mice (FIG. 1A). Luminex measurement of serum cytokines showed the typical delayed Th1 (IFN-γ, IL-2 and IL-12p40) and an absence of Th2 response along the course of infection (FIG. 1B). The above results are consistent with the observation in patients infected with HCV (Fahey S, Dempsey E, Long A. The role of chemokines in acute and chronic hepatitis C infection. Cellular and Molecular Immunology 11, 25 (2014)).

### Example 2. Blockade of T Cell Immune Checkpoint has No Effect on Chronic Infection with HCV

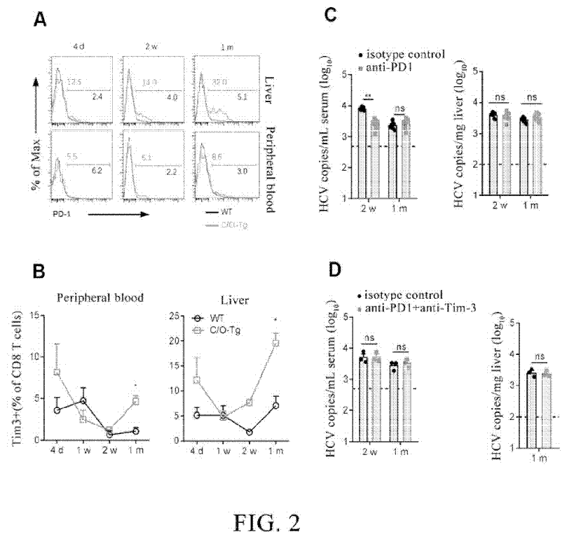

Since CD8+ T cells are generally thought to play an important role in viral infection and clearance, the expression profile of CD8+T immune checkpoint molecules during HCV infection was first tested. It was found that the T cell immune checkpoint molecules PD-1 (FIG. 2A) and Tim-3 (FIG. 2B) were up-regulated with the establishment of chronic infection after infection of mice with HCV. Based on this, it was tested whether targeting T cell immune checkpoint molecules can inhibit the chronic infection process. However, as shown in the results of FIGS. 2C and 2D, PD-1 blocking antibody (clone No. G4, produced by hybridoma) alone or combination of PD-1 and Tim-3 blocking antibodies (clone No. BE0115, purchased from BioXcell) cannot effectively promote virus elimination. The above results indicate that targeting T cell immune checkpoint molecules cannot effectively inhibit the chronic infection process of HCV, nor promote the elimination of HCV.

### Example 3. Depletion of NK Cells Leads to Persistent Infection with HCV

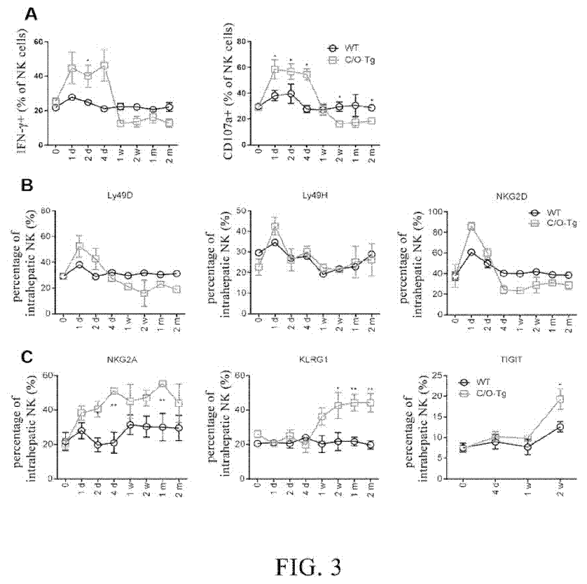

The effect of HCV infection on NK cell function was subsequently tested. Depletion of liver NK cells during HCV infection was tested by an in vitro NK function assay. The results showed that the liver NK cells of HCV-infected C/O-Tg mice were stimulated by the target cell Yac-1, and the IFN-γ secretion capacity and CD107a degranulation level increased within four days after HCV infusion, which subsequently rapidly decreased to a baseline level similar to uninfected liver NK cells (FIG. 3A). Further studies showed up-regulation of NK activation receptors Ly49D, Ly49H, and NKG2D within 4 days after HCV infusion (FIG. 3B). These results show transient liver infiltration and activation of NK cells in response to HCV infection. However, these activation receptors decreased in liver NK cells four days after HCV infusion, while the expression of immune checkpoint molecules KLRG1, NKG2A, and TIGIT increased and maintained at two months after HCV infusion (FIG. 3C).

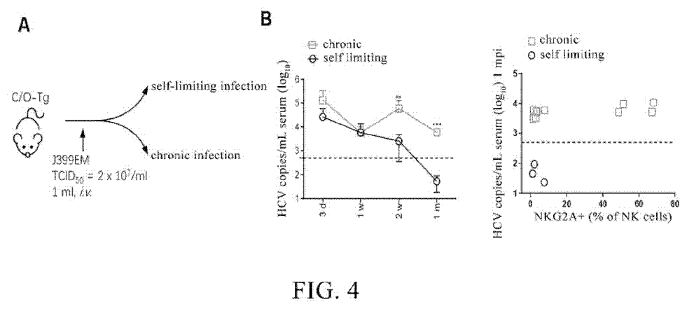

In addition, spontaneous elimination of HCV was observed in a small portion of HCV-infected C/O-Tg mice forming a self-limiting infection (FIG. 4A). Therefore, the depletion of NK cells and the expression profiles of immune checkpoint molecules in C/O-Tg mouse populations with different HCV infection results (i.e. self-limiting infection or chronic infection) were further investigated. It was found that NK cells in mice capable of spontaneously eliminating HCV showed very low NKG2A expression, while mice that developed persistent HCV infection showed higher levels of NKG2A (FIG. 4B). The above results suggest that during the conversion of HCV infection from acute infection phase to chronic infection phase, the up-regulation of immune checkpoint molecules including NKG2A affects the function of NK cells and causes their depletion, which promotes the development of HCV infection from acute infection phase to chronic phase, leading to a persistent infection.

### Example 4. Blockade or Inhibition of NKG2A Promotes Elimination of HCV

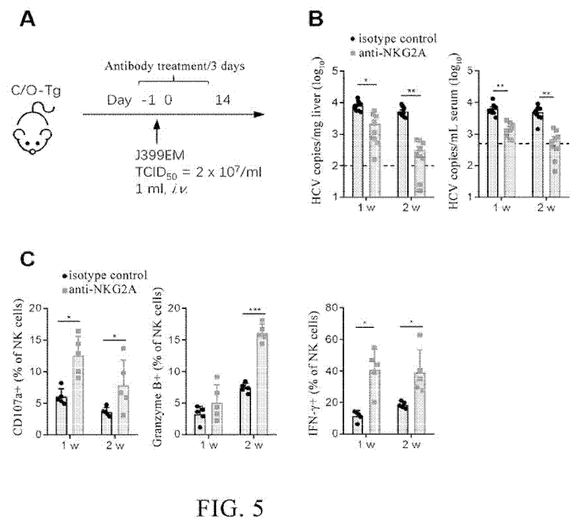

Since NKG2A expression is up-regulated in mice that develop persistent infection with HCV, the use of antagonists to block NKG2A for the prevention and treatment of HCV infection was further tested in the present disclosure. First, C/O-Tg mice were treated with NKG2A blocking antibody (clone No. 20D5, purchased from Them or control antibody (50 μg/time, intraperitoneal injection) at the time when injecting HCV (J399EM, 1 mL, TCID₅₀=2×10⁷) into the mice (n=9 per group), and the antibody was further administered every 3 days for one or two weeks after HCV infusion (FIG. 5A). The results showed that compared with the isotype control antibody treated group, the viral load in the serum and liver of mice in the NKG2A blocking antibody treated group decreased significantly at one and two weeks (FIG. 5B). The decrease in HCV copy number after anti-NKG2A treatment was associated with increased killing activity of NK cell and enhanced IFN-γ secretion capacity of NK cell (FIG. 5C).

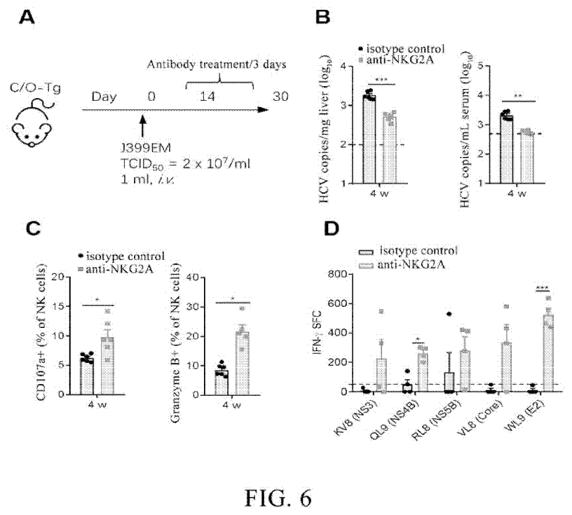

In order to further investigate the therapeutic effect of NKG2A blockade on chronic HCV infection, mice were administered with NKG2A blocking antibodies two weeks after HCV infection. Up-regulation of NKG2A expression has been observed at this stage (FIG. 3C) and administration was lasted for two weeks (FIG. 6A). The results showed that blockade of NKG2A reduced viral levels in the liver and serum (FIG. 6B), and it was associated with increased liver NK cell activity (FIG. 6C) and HCV-specific T cell response (FIG. 6D), which is demonstrated by the IFN-γ secretion levels after stimulation of T cells with HCV peptides NS3, NS4B, NS5B, Core and E2. The above results show that targeting NKG2A can break the response tolerance of NK cells and HCV-specific T cells and play a role in eliminating viruses.

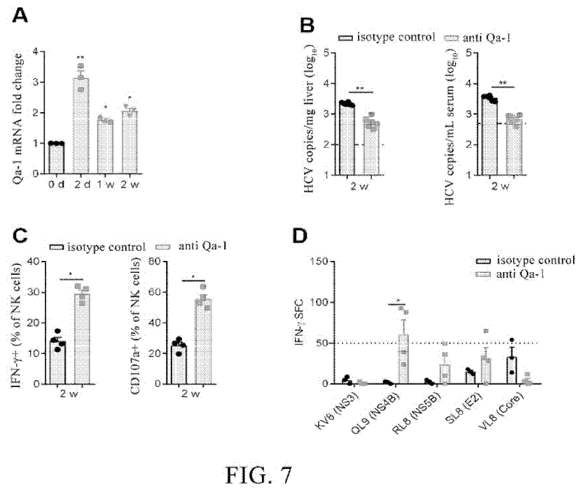

Since HLA-E in human or Qa-1 in mouse is the ligand interacting with NKG2A to limit NK function, the expression profile of the ligand of NKG2A during HCV infection was further tested. By detection of the transcription level of Qa-1 in liver tissues at different time points after infection of mice with HCV, it was found that Qa-1 was mainly expressed in liver parenchymal cells rather than immune cells, and Qa-1 expression level was significantly up-regulated after HCV infection (FIG. 7A). Therefore, it was further tested whether antagonism using Qa-1 antibodies can inhibit or reverse the depletion of NK cells. The results showed that compared to the control group, administration of Qa-1 blocking antibody (clone No. 6A8.6F10.1A6, purchased from BD Pharmigen) inhibited HCV replication in HCV-infected C/O-Tg mice (FIG. 7B), accompanied by the restoration of NK cell function (FIG. 7C) and restoration of CD8+ T cell function (FIG. 7D).

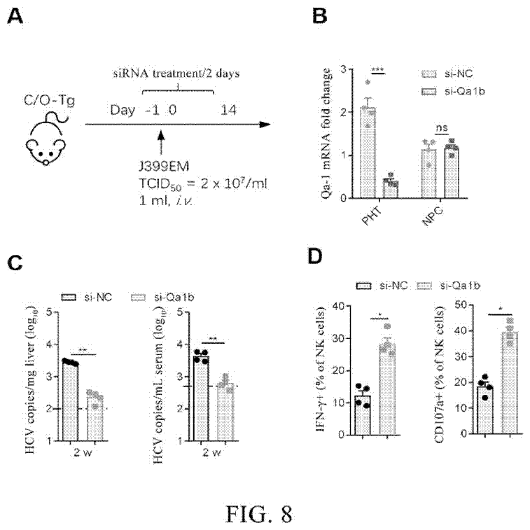

On the other hand, it was investigated whether suppression of the expression of immune checkpoint molecules can promote the elimination of HCV. In this regard, cholesterol-conjugated siRNA against Qa-1 (sequence: GAAGAGGAGGAGACACAUA, synthesized by GenePharma) was delivered via tail vein injection in HCV-infected C/O-Tg mice (FIG. 8A), which selectively down-regulated Qa-1 in hepatocytes (FIG. 8B). The results showed that down-regulation of Qa-1 expression on hepatocytes inhibited HCV replication (FIG. 8C) and reversed the depletion process of NK cells (FIG. 8D). In summary, these results indicate that the interaction of the immune checkpoint molecules Qa-1/NKG2A impairs NK cell function and leads to its depletion, which promotes the establishment of persistent HCV infection, and targeting the immune checkpoint molecules as described above can reverse the process and promote elimination of viruses.

### Example 5. Targeting NKG2A on NK Cells Instead of T Cells Promotes HCV Elimination

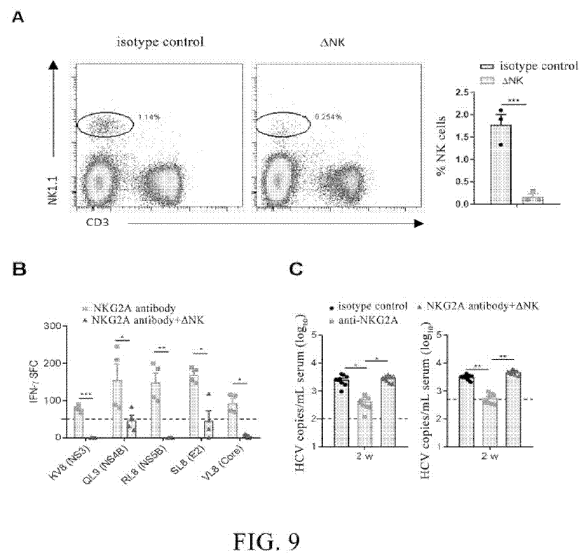

Since NKG2A is also expressed in a portion of T cells, it was further investigated that whether the expression of NKG2A on NK cells or T cells was essential for the establishment of persistent HCV infection. To verify the role of NK cells in this process, NK cells were deleted in HCV-infected C/O-Tg mice before treatment with anti-NKG2A (FIG. 9A). The results showed that in the absence of NK cells, blocking NKG2A with antibodies failed to restore the activity of HCV-specific T cells (FIG. 9B), and the levels of HCV virus were significantly increased in mice (FIG. 9C). In the presence of NK cells, NKG2A antibodies can restore activity of HCV-specific T cells and promote HCV elimination. Therefore, restoration of the cytotoxicity of HCV-specific T cells by anti-NKG2A depends on NK cells.

1.-19. (canceled)

20. A method for blocking, inhibiting, and/or reversing NK cell depletion in a subject, wherein the subject has or is at risk of suffering from an infectious disease caused by viral infection, the method comprising the step of administering to the subject an effective amount of an antagonist or expression inhibitor against an NK cell immune checkpoint molecule.

21. The method of claim 20, wherein the virus is selected from the group consisting of human immunodeficiency virus (HIV), hepatitis B virus (HBV), and hepatitis C virus (HCV).

22. The method of claim 20, wherein the infectious disease is in a chronic infection phase.

23. The method of claim 20, wherein the NK cell immune checkpoint molecule is selected from the group consisting of KIR, NKG2A, TIGIT and KLRG1.

24. The method of claim 20, wherein the antagonist is an antibody or antigen-binding fragment thereof against the immune checkpoint molecule or a soluble form of a corresponding ligand/receptor or a fragment thereof of the immune checkpoint molecule, or the expression inhibitor is microRNA or siRNA.

25. The method of claim 24, wherein the NK cell immune checkpoint molecule is NKG2A, and the antagonist is an antibody or an antigen-binding fragment thereof directed against NKG2A or its ligand HLA-E or is a soluble form of HLA-E or a fragment thereof.

26. The method of claim 20, wherein the subject is a human or non-human primate.

27. The method of claim 20, wherein the subject is not responsive or is tolerant to a DAA drug treatment.

28. The method of claim 20, further comprising the step of administering one or more additional therapeutic agents to the subject, wherein the additional therapeutic agent is selected from an NK cell activation agent or is a direct-acting antiviral (DAA) drug.

29. The method of claim 28, wherein the NK cell activation agent is an agonist for an NK cell activating receptor, an antagonist for an NK cell inhibitory receptor, or a cytokine or chemokine that activates NK cells or wherein the virus is HCV, and the DAA drug is selected from the group consisting of Telaprevir, Boceprevir, Simeprevir, Asunaprevir, Sofosbuvir, Mericitabine (RG-7128), ACH-3422, MK-3682 and Daclatasvir.

30. A method of preventing or treating an infectious disease caused by viral infection in a subject, the method comprising the step of administering to the subject an effective amount of an antagonist or expression inhibitor against an NK cell immune checkpoint molecule.

31. The method of claim 30, wherein the virus is selected from the group consisting of human immunodeficiency virus (HW), hepatitis B virus (HBV), and hepatitis C virus (HCV).

32. The method of claim 30, wherein the infectious disease is in a chronic infection phase.

33. The method of claim 30, wherein the NK cell immune checkpoint molecule is selected from the group consisting of KIR, NKG2A, TIGIT and KLRG1.

34. The method of claim 30, wherein the antagonist is an antibody or antigen-binding fragment thereof against the immune checkpoint molecule or a soluble form of a corresponding ligand/receptor or a fragment thereof of the immune checkpoint molecule, or the expression inhibitor is microRNA or siRNA.

35. The method of claim 34, wherein the NK cell immune checkpoint molecule is NKG2A, and the antagonist is an antibody or antigen-binding fragment thereof directed against NKG2A or its ligand HLA-E or is a soluble form of HLA-E or a fragment thereof.

36. The method of claim 30, wherein the subject is a human or non-human primate.

37. The method of claim 30, wherein the subject is not responsive or is tolerant to DAA drug treatment.

38. The method of claim 30, further comprising the step of administering one or more additional therapeutic agents to the subject, wherein the additional therapeutic agent is selected from an NK cell activation agent or is a direct-acting antiviral (DAA) drug.

39. The method of claim 38, wherein the NK cell activation agent is an agonist for an NK cell activating receptor, an antagonist for an NK cell inhibitory receptor, or a cytokine or chemokine that activates NK cells or wherein the virus is HCV, and the DAA drug is selected from the group consisting of Telaprevir, Boceprevir, Simeprevir, Asunaprevir, Sofosbuvir, Mericitabine (RG-7128), ACH-3422, MK-3682 and Daclatasvir.
